(19) United States
(12) Patent Application Publication
(10) Pub. No.: US 2014/0186830 A1
(43) Pub. Date: Jul. 3, 2014

(54) Methods for Analyzing Short Tandem Repeats and Single Nucleotide Polymorphisms

(71) Applicant: Applied Biosystems, LLC, Carlsbad, CA (US)

(72) Inventor: Lori K. HENNESSY, San Mateo, CA (US)

(73) Assignee: Applied Biosystems, LLC, Carlsbad, CA (US)

(21) Appl. No.: 14/012,943

(22) Filed: Aug. 28, 2013

Related U.S. Application Data
(63) Continuation of application No. 11/171,492, filed on Jun. 29, 2005, now abandoned.
(60) Provisional application No. 60/584,774, filed on Jun. 30, 2004.

(51) Int. Cl. C12Q 1/68

(52) U.S. Cl. CPC C12Q 1/6827
USPC 435/6.11

(57) ABSTRACT

Methods for genotyping a sample comprising nucleic acid are provided. The methods comprise analyzing STR and SNP loci.

This application claims the benefit of U.S. Provisional Application No. 60/584,774, filed Jun. 30, 2004, which is incorporated by reference herein in its entirety for any purpose.

### I. FIELD

Methods for genotyping are provided which analyze STR and SNP loci.

### II. BACKGROUND

Short tandem repeats (STRs), also called microsatellites, are tandemly repeated units of DNA distributed throughout the human genome. (For a review of STRs, see, e.g., Hohoff et al. (1999) Mol. Biotech. 13:123-138.) The repeated units are typically of two to seven base pairs. In certain instances, the size of an STR may be hundreds of base pairs, depending on the number of repeated units. The number of repeated units varies among individuals. The polymorphic nature of STRs allows them to be used in various methods, including genetic linkage studies, forensic DNA typing, and clinical diagnostics.

SNPs, or single nucleotide polymorphisms, are also a source of genetic variation among individuals. SNPs occur throughout the genome and may be used in various methods, including genetic linkage studies, forensic DNA typing, and clinical diagnostics.

### III. SUMMARY

In certain embodiments, a method of genotyping a sample comprising nucleic acid is provided. In certain embodiments, the method comprises analyzing a plurality of STR loci in the sample and analyzing at least one SNP locus in the sample, thereby genotyping the sample. In certain embodiments, the plurality of STR loci composes one or more CODIS STR loci, in certain embodiments, the analyzing a plurality of STR loci comprises using PCR to generate a plurality of PCR products. In certain embodiments, the size of at least two of the plurality of PCR products indicates the identity of at least two STR alleles.

In certain embodiments, the analyzing a plurality of STR loci comprises: combining at least a portion of the sample with a plurality of STR-specific primer sets, wherein an STR-specific primer set comprises a first primer and a second primer for amplifying an STR locus; and subjecting the sample to amplification. In certain embodiments, at least one of the primers in the plurality of STR-specific primer sets further comprises a label.

In certain embodiments, the analyzing a plurality of STR loci and the analyzing at least one SNP locus comprise processes that occur in separate reaction mixtures. In certain embodiments, the analyzing a plurality of STR loci and the analyzing at least one SNP locus further comprise combining the separate reaction mixtures to form a combined reaction mixture. In certain embodiments, the analyzing a plurality of STR loci and the analyzing at least one SNP locus further comprise detecting in the combined reaction mixture one or more labels that Identify a plurality of STR alleles and at least one SNP allele in a single output.

In certain embodiments, the at least one SNP locus provides information on phenotype. In certain embodiments, the analyzing at least one SNP locus comprises combining at least a portion of the sample with at least one allele-specific primer and subjecting the at least a portion of the sample to an extension assay. In certain embodiments, the analyzing at least one SNP locus comprises using allele-specific PCR or an allele-specific primer extension assay. In certain embodiment, the analyzing at least one SNP locus comprises using an allele-specific nucleotide incorporation assay. In certain embodiments, the analyzing at least one SNP locus comprises using a single base extension assay.

In certain embodiments, the analyzing at least one SNP locus comprises combining at least a portion of the sample with at least one allele-specific probe and detecting hybridization of the at least one allele-specific probe to the SNP locus. In certain embodiments, the analyzing at least one SNP locus comprises using a method selected from an allele-specific oligonucleotide hybridization assay, a 5′ nuclease assay, an assay employing molecular beacons, an assay employing flap endonuclease, and an oligonucleotide ligation assay.

In certain embodiments, the analyzing a plurality of STR loci and the analyzing at least one SNP locus occur in the same reaction mixture. In certain embodiments, the analyzing a plurality of STR loci and the analyzing at least one SNP locus comprise using PCR. In certain embodiments, the analyzing at least one SNP locus comprises using allele-specific PCR.

In certain embodiments, a kit for analyzing a plurality of STR loci and at least one SNP locus in a sample comprising nucleic acid is provided. In certain embodiments, the kit comprises a plurality of STR-specific primer sets and at least one primer that selectively hybridizes to a SNP locus. In certain embodiments, the kit further comprises at least one universal primer comprising a label. In certain embodiments, the at least one primer that selectively hybridizes, to a SNP locus is an allele-specific primer. In certain embodiments, the plurality of STR-specific primer sets and the at least one primer that selectively-hybridizes to a SNP locus are capable of generating detectable amplication products in a single reaction mixture, wherein the amplification products indicate the identity of a plurality of STR alleles and at least one SNP allele. In certain embodiments, the plurality of STR-specific primer sets and the at least one primer that selectively hybridizes to a SNP locus generate amplification products that are detectable in a single output, wherein the amplification products indicate the identify of a plurality of STR alleles and at least one SNP allele. In certain embodiments, amplification products from different loci do not overlap in size. In certain embodiments, amplification products from different loci overlap in size. In certain such embodiments, amplication products that overlap in size further comprise different labels.

In certain embodiments, the kit comprises a plurality of STR-specific primer sets and at least one probe that selectively hybridizes to a SNP locus. In certain embodiments, the kit further comprises at least one universal primer comprising a label. In certain embodiments, the at least one probe that selectively hybridizes to a SNP locus is an allele-specific probe. In certain embodiments, the at least one probe that selectively hybridizes to a SNP locus comprises at least one allele-specific probe and a second probe suitable for use in an oligonucleotide ligation assay. In certain embodiments, the plurality of STR-specific primer sets and the at least one probe that selectively hybridizes to a SNP locus allow identification of a plurality of STR alleles and at least one SNP allele in a single output.

### IV. BRIEF DESCRIPTION OF THE DRAWINGS

FIG. 1A and 1B shows certain exemplary embodiments of analyzing a plurality of STR loci and at least one SNP locus in a single reaction mixture. In the embodiments shown in FIG. 1A, three STR loci are amplified by PCR in a reaction mixture. Two SNP loci are subjected to allele-specific PCR in the same reaction mixture. FIG. 1B shows an example of an output that may result from capillary electrophoresis (CE) of at least a portion of the reaction mixture.

FIG. 2 shows certain exemplary embodiments of analyzing a plurality of STR loci and at least one SNP locus. In the embodiments shown In FIG. 2, the analyzing comprises amplifying the STR loci and the at least one SNP locus in separate reaction mixtures, followed by combining at least a portion of the reaction mixtures and detecting amplification products using capillary electrophoresis.

### V. DETAILED DESCRIPTION OF CERTAIN EXEMPLARY EMBODIMENTS

It is to be understood that both the foregoing general description and the following detailed description are exemplary and explanatory only and are not restrictive of the invention, as claimed. In this application, the use of the singular includes the plural unless specifically stated otherwise. In this application, the word “a” or “an” means “at least one” unless specifically stated otherwise. In this application, the use of “or” means “and/or” unless stated otherwise. Furthermore, the use of the term “including,” as well as other forms, such as “includes” and “included,” is not limiting. Also, terms such as “element” or “component” encompass both elements or components comprising one unit and elements or components that comprise more than one unit unless specifically stated otherwise.

The section headings used herein are for organizational purposes only and are not to be construed as limiting the subject matter described. All documents, or portions of documents, cited in this application, including but not limited to patents, patent applications, articles, books, and treatises, are hereby expressly incorporated by reference in their entirety for any purpose.

### A. CERTAIN DEFINITIONS

The term “nucleotide base,” as used herein, refers to a substituted or unsubstituted aromatic ring or rings. In certain embodiments, the aromatic ring or rings contain at least one nitrogen atom. In certain embodiments, the nucleotide base is capable of forming Watson-Crick and/or Hoogsteen hydrogen bonds with an appropriately complementary nucleotide base. Exemplary nucleotide bases and analogs thereof include, but are not limited to, naturally occurring nucleotide bases adenine, guanine, cytosine, 6 methyl-cytosine, uracil, thymine, and analogs of the naturally occurring nucleotide bases, e.g., 7-deazaadenine, 7-deazaguanine, 7-deaza-8-azaguanine, 7-deaza-8-azaadenine, N6-Δ2-isopentenyladenine (6iA), N6-Δ2-isopentenyl-2-methylthioadenine (2ms6iA), N2-dimethylguanine (dmG), 7-methylguanine (7mG), inosine, nebularine, 2-aminopurine, 2-amino-6-chloropurine, 2,6-diaminopurine, hypoxanthine, pseudouridine, pseudocytosine, pseudoisocytosine, 5-propyoylcytosine, isocytosine, isoguanine, 7-deazaguanine, 2-thiopyrimidine, 6-thioguanine, 4-thiothymine, 4-thiouracil, O⁶-methylguanine, N⁶-methyladenine, O⁴-methylthymine, 5,6-dihydrothymine, 5,6-dihydrouracil, pyrazolo[3,4-D]pyrimidines (see, e.g., U.S. Pat. Nos. 6,143,877 and 6,127,121 and PCT published application WO 01/38684), ethenoadenine, indoles such as nitroindole and 4-methylindole, and pyrroles such as nitropyrrole. Certain exemplary nucleotide bases can be found, e.g., in Fasman, 1989, Practical Handbook of Biochemistry and Molecular Biology, pp. 385-394, CRC Press, Boca Raton, Fla., and the references cited therein.

The term “nucleotide,” as used herein, refers to a compound comprising a nucleotide base linked to the C-1′ carbon of a sugar, such as ribose, arabinose, xylose, and pyranose, and sugar analogs thereof. The term nucleotide also encompasses nucleotide analogs. The sugar may be substituted or unsubstituted. Substituted ribose sugars include, but are not limited to, those riboses in which one or more of the carbon atoms, for example the 2′-carbon atom, is substituted with one or more of the same or different Cl, F, —R, —OR, —NR₂ or halogen groups, where each R is independently H, C₁-C₆ alkyl or C₅-C₁₄ aryl. Exemplary riboses include, but are not limited to, 2═-(C1-C6)alkoxyriboses 2′-(C5-C14)aryloxyribose, 2′,3′-didehydroribose, 2′-deoxy-3′-haloribose, 2′-deoxy-3′-fluororibose, 2′-deoxy-3′-chlororibose, 2′-deoxy-3′-aminoribose, 2′-deoxy-3′-(C1-C6)alkylribose, 2′-deoxy-3′-(C1-C6)alkoxyribose and 2′-deoxy-3′-(C5-C14)aryloxyribose, ribose, 2′-deoxyribose, 2′,3′-dideoxyribose, 2′-haloribose, 2′-fluororibose, 2′-chlororibose, and 2′-alkylribose, e.g., 2′-O-methyl, 4′-α-anomeric nucleotides, 1′-α-anomeric nucleotides, 2′-4′- and 3′-4′-linked and other “locked” or “LNA”, bicyclic sugar modifications (see, e.g., PCT published application nos. WO 98/22489, WO 98/39362, and WO 99/14228). Exemplary LNA sugar analogs within a polynucleotide include, but are not limited to, the structures:

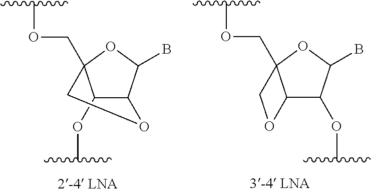

where B is any nucleotide base.

Modifications at the 2′- or 3′-position of ribose include, but are not limited to, hydrogen, hydroxy, methoxy, ethoxy, allyloxy, isopropoxy, butoxy, isobutoxy, methoxyethyl, alkoxy, phenoxy, azido, amino, alkylamino, fluoro, chloro and bromo. Nucleotides include, but are not limited to, the natural D optical isomer, as well as the L optical isomer forms (see, e.g., Garbesi (1993) Nucl. Acids Res. 21:4159-65; Fujimori (1990) J. Amer. Chem. Soc. 112:7435; Urata, (1993) Nucleic Acids Symposium Ser. No. 29:69-70). When the nucleotide base is purine, e.g. A or G, the ribose sugar is attached to the N⁹-position of the nucleotide base. When the nucleotide base is pyrimidine, e.g. C, T or U, the pentose sugar is attached to the N¹-position of the nucleotide base, except for pseudouridines, in which the pentose sugar is attached to the C5 position of the uracil nucleotide base (see, e.g., Kornberg and Baker, (1992) DNA Replication, 2^{nd} Ed., Freeman, San Francisco, Calif.).

One or more of the pentose carbons of a nucleotide may be substituted with a phosphate ester having the formula:

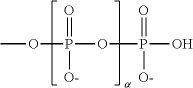

where α is an integer from 0 to 4. In certain embodiments, α is 2 and the phosphate ester is attached to the 3′- or 50′-carbon of the pentose. In certain embodiments, the nucleotides are those in which the nucleotide base is a purine, a 7-deazapurine, a pyrimidine, or an analog thereof. “Nucleotide 5′-triphosphate” refers to a nucleotide with a triphosphate ester group at the 5′ position, and is sometimes denoted as “NTP”, or “dNTP” and “ddNTP” to particularly point out the structural features of the ribose sugar. The triphosphate ester group may include sulfur substitutions for the various oxygens, e.g., α-thio-nucleotide 5′-triphosphates. For a review of nucleotide chemistry, see: Shabarova, Z., and Bogdanov, A. Advanced Organic Chemistry of Nucleic Acids, VCH, New York, 1994.

The term “nucleotide analog,” as used herein, refers to embodiments in which the pentose sugar and/or the nucleotide base and/or one or more of the phosphate esters of a nucleotide may be replaced with its respective analog. In certain embodiments, exemplary pentose sugar analogs are those described above. In certain embodiments, the nucleotide analogs have a nucleotide base analog as described above. In certain embodiments, exemplary phosphate ester analogs include, but are not limited to, alkylphosphonates, methylphosphonates, phosphoramidates, phosphotriesters, phosphorothioates, phosphorodithioates, phosphoroselenoates, phosphorodiselenoates, phosphoroanilothioates, phosphoroanilidates, phosphoroamidates, boronophosphates, etc., and may include associated counterions.

Also included within the definition of “nucleotide analog” are nucleotide analog monomers that can be polymerized into polynucleotide analogs in which the DNA/RNA phosphate ester and/or sugar phosphate ester backbone is replaced with a different type of internucleotide linkage. Exemplary polynucleotide analogs include, but are not limited to, peptide nucleic acids, in which the sugar phosphate backbone of the polynucleotide is replaced by a peptide backbone.

As used herein, the terms “polynucleotide,” “oligonucleotide,” and “nucleic acid” are used interchangeably and mean single-stranded and double-stranded polymers of nucleotide monomers, including 2′-deoxyribonucleotides (DNA) and ribonucleotides (RNA) linked by internucleotide phosphodiester bond linkages, or internucleotide analogs, and associated counter ions, e.g., H⁺, NH₄⁺, trialkylammonium, Mg²⁺, Na⁺ and the like. A nucleic acid may be composed entirely of deoxyribonucleotides, entirely of ribonucleotides, or chimeric mixtures thereof. The nucleotide monomer units may comprise any of the nucleotides described herein, including, but not limited to, naturally occurring nucleotides and nucleotide analogs. Nucleic acids typically range in size from a few monomeric units, e.g. 5-40 when they are sometimes referred to in the art as oligonucleotides, to several thousands of monomeric nucleotide units. Unless denoted otherwise, whenever a nucleic acid sequence is represented, it will be understood that the nucleotides are in 5′ to 3′ order from left to right and that “A” denotes deoxyadenosine or an analog thereof, “C” denotes deoxycytidine or an analog thereof, “G” denotes deoxyguanosine or an analog thereof, “T” denotes thymidine or an analog thereof, and “U” denotes uridine or an analog thereof, unless otherwise noted.

Nucleic acids include, but are not limited to, genomic DNA, cDNA, hnRNA, mRNA, rRNA, tRNA, fragmented nucleic acid, nucleic acid obtained from subcellular organelles such as mitochondria or chloroplasts, and nucleic acid obtained from microorganisms or DNA or RNA viruses that may be present on or in a biological sample. Nucleic acids include, but are not limited to, synthetic or in vitro transcription products.

Nucleic acids may be composed of a single type of sugar moiety, e.g., as in the case of RNA and DNA, or mixtures of different sugar moieties, e.g., as in the case of RNA/DNA chimeras. In certain embodiments, nucleic acids are ribopolynucleotides and 2′-deoxyribopolynucleotides according to the structural formulae below:

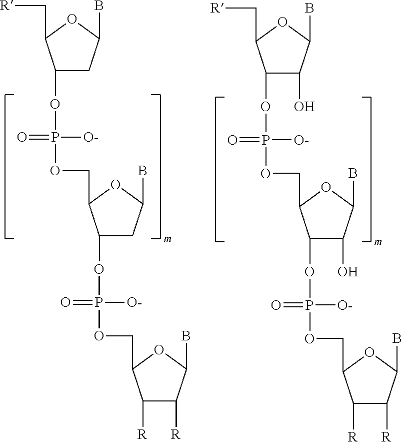

wherein each B is independently the base moiety of a nucleotide, e.g., a purine, a 7-deazapurine, a pyrimidine, or an analog nucleotide; each m defines the length of the respective nucleic acid and can range from zero to thousands, tens of thousands, or even more; each R is independently selected from the group comprising hydrogen, halogen, —R″. —OR″, and —NR″R″, where each R″ is independently (C1-C6) alkyl or (C5-C14) aryl, or two adjacent Rs are taken together to form a bond such that the ribose sugar is 2′,3′-didehydroribose; and each R′ is independently hydroxyl or

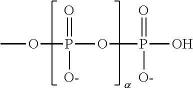

where α is zero, one or two.

In certain embodiments of the ribopolynucleotides and 2′-deoxyribopolynucleotides illustrated above, the nucleotide bases B are covalently attached to the C1′ carbon of the sugar moiety as previously described.

The terms “nucleic acid,” “polynucleotide,” and “oligonucleotide” may also include nucleic acid analogs, polynucleotide analogs, and oligonucleotide analogs. The terms “nucleic acid analog”, “polynucleotide analog” and “oligonucleotide analog” are used interchangeably and, as used herein, refer to a nucleic acid that contains at least one nucleotide analog and/or at least one phosphate ester analog and/or at least one pentose sugar analog. Also included within the definition of nucleic acid analogs are nucleic acids in which the phosphate ester and/or sugar phosphate ester linkages are replaced with other types of linkages, such as N-(2-aminoethyl)-glycine amides and other amides (see, e.g., Nielsen et al., 1991, Science 254:1497-1500; WO 92/20702; U.S. Pat. No. 5,719,262; U.S. Pat. No. 5,698,685;); morpholinos (see, e.g., U.S. Pat. No. 5,698,685; U.S. Pat. No. 5,378,841; U.S. Pat. No. 5,185,144); carbamates (see, e.g., Stirchak & Summerton, 1987, J. Org. Chem. 52; 4202); methylene(methylimino) (see, e.g., Vasseur et al., 1992, J. Am. Chem. Soc. 114:4006); 3′-thioformacetals (see, e.g. Jones et al., 1993, J. Org. Chem. 58; 2983); sulfamates (see, e.g., U.S. Pat. No. 5,470,967); 2-aminoethylglycine, commonly referred to as PNA (see, e.g., Buchardt, WO 92/20702; Nielsen (1991) Science 254:1497-1500); and others (see, e.g., U.S. Pat. No. 5,817,781; Frier & Altman, 1997, Nucl. Acids Res. 25:4429 and the references cited therein). Phosphate ester analogs include, but are not limited to, (i) C₁-C₄ alkylphosphonate, e.g. methylphosphonate; (ii) phosphoramidate; (iii) C₁-C₆ alkyl-phosphotriester; (iv) phosphorothioate; and (v) phosphorodithioate,

The term “analyzing,” in reference to an STR locus, refers to carrying out one or more processes for identifying the STR allele present at the STR locus. An “STR locus” refers to a region of a chromosome containing repeated units that vary in number among certain individuals of a given species, such as humans. The term “STR locus” encompasses a copy of such a chromosomal region produced, for example, by an amplification reaction.

The term “CODIS STR loci” as used herein refers to the thirteen core STR loci designated by the FBI's “Combined DMA Index System.” The thirteen core STR loci are TH01, TPOX, CSF1PO, vWA, FGA, D3S1358, D5S818, D7S820, D13S317, D18S539, D8S1179, D18S51, and D21S11. (See, e.g., Butler, Forensic DNA Typing, Academic Press (2001), at page 63.)

The term “analyzing,” in reference to a SNP locus, refers to carrying out one or more process for identifying the SNP allele present at a SNP locus. In certain embodiments, identifying the SNP allele present at a SNP locus comprises identifying the nucleotide at a polymorphic site as an A, C, G, or T. The term “SNP locus” refers to a region of a chromosome comprising a nucleotide that differs among certain individuals of a given species, such as humans. The term “SNP locus” encompasses a copy of such a chromosomal region produced, for example, by an amplification reaction. The term “polymorphic site” as used herein refers to at least one nucleotide site in a DNA sequence that differs among certain individuals of a given species, such as humans.

The term “allele-specific primer” refers to a polynucleotide that selectively hybridizes to a SNP locus at a region comprising a polymorphic site. An allele-specific primer comprises a “pivotal nucleotide” that is complementary to one of the possible nucleotides at a polymorphic site. In the presence of a polymerase, nucleotides may be added to the 3′ end of an allele-specific primer.

The term “allele-specific probe” refers to a polynucleotide that selectively hybridizes to a SNP locus at a region comprising a polymorphic site. An allele-specific probe comprises a “pivotal nucleotide” that is complementary to one of the possible nucleotides at the polymorphic site.

A primer that “selectively hybridizes to a SNP locus” refers to a primer that selectively hybridizes to a SNP locus at a region that comprises a polymorphic site or at a region that is 3′ of a polymorphic site. In the presence of a polymerase, nucleotides may be added to the 3′ end of a primer that selectively hybridizes to a SNP locus.

A probe that “selectively hybridizes to a SNP locus” refers to a probe that selectively hybridizes to a SNP locus at a region that comprises a polymorphic site or at a region that is either 5′ or 3′ of a polymorphic site.

The term “extension assay” refers to an assay in which nucleotides are added to a nucleic acid, resulting in a longer nucleic acid. The term “extension product” refers to the resultant longer nucleic acid. A non-limiting exemplary extension assay is one that employs a polymerase to add one or more nucleotides to the 3′ end of a primer. Exemplary extension assays include, but are not limited to, primer extension assays, including allele-specific primer extension assays; PCR, including allele-specific PCR; and allele-specific nucleotide incorporation assays.

The term “allele-specific primer extension assay” refers to an extension assay in which a SNP locus is combined with one or more allele-specific primers. When more than one allele-specific primer is used, the allele-specific primers may comprise different pivotal nucleotides. In a non-limiting exemplary allele-specific primer extension assay, the pivotal nucleotides are present at the 3′ ends of the allele-specific primers. A polymerase is used to add one or more nucleotides to the 3′ ends of the allele-specific primers if the primers are appropriately hybridized to the SNP locus. For non-limiting examples of allele-specific primer extension assays, see, e.g., Ye et al. (2001) Hum. Mut. 17:305-316; and Shen et al. Genetic Engineering News, vol. 23, Mar. 15, 2003.

The term “allele-specific PCR” refers to an extension assay in which a SNP locus is amplified by the polymerase chain reaction. The reaction comprises one or more allele-specific primers that comprise different pivotal nucleotides. In a non-limiting example of allele-specific PCR, the pivotal nucleotides are present at the 3′ ends of the allele-specific primers. For a non-limiting example of allele-specific PCR, see, e.g., McClay et al. (2002) Analytical Biochem. 301:200-206.

The term “allele-specific nucleotide incorporation assay” refers to an extension assay in which a primer selectively hybridizes to a SNP locus at a region that is 3′ of a polymorphic site. At least one nucleotide is then added to the 3′ end of the primer by a polymerase, such that a nucleotide that is complementary to the nucleotide at the polymorphic site is incorporated into the growing polynucleotide. Exemplary allele-specific nucleotide incorporation assays include, but are not limited to, single base extension assays.

A “single base extension assay” or a “single base chain extension assay” refers to an extension assay in which a primer selectively hybridizes to a SNP locus at a region that is immediately 3′ of a polymorphic site. A single nucleotide that is complementary to the nucleotide at the polymorphic site is then added to the 3′ end of the primer by a polymerase. For non-limiting examples of single base extension assays, see, e.g., Chen et al. (2000) Genome Res. 10:549-557; Fan et al. (2000) Genome Res. 10:853-860; Pastinen et al. (1997) Genome Res. 7:606-614; and Ye et al. (2001) Hum. Mut. 17:305-316.

The term “allele-specific oligonucleotide hybridization assay” refers to an assay which detects hybridization between at least one polynucleotide comprising a polymorphic site and at least one oligonucleotide comprising a nucleotide that is complementary to the polymorphic site. For non-limiting examples of allele-specific oligonucleotide hybridization assays, see, e.g., Saiki et al. (1989) Proc. Nat'l Acad. Sci. USA 86:8230-8234; and Wang et al. (1998) Science 280:1077-1082.

The term “5′ nuclease assay” refers to an assay in which a SNP locus is combined with one or more allele-specific probes. When more than one allele-specific probe is used, the allele-specific probes may comprise different pivotal nucleotides. The SNP locus and the allele-specific probes are further combined with a polymerase having 5′ nuclease activity and with one or more primers that are capable of amplifying a region that comprises the region to which the allele-specific probes hybridize. The SNP locus is then subjected to amplification. Allele-specific probes comprising a pivotal nucleotide that is complementary to the polymorphic site will be cleaved by the polymerase during amplification. Allele-specific probes comprising a pivotal nucleotide that is not complementary to the polymorphic site will not be substantially cleaved by the polymerase during amplification. In certain embodiments of 5′ nuclease assays, the allele-specific probe includes a fluorescent molecule and a quenching molecule. When the probe is cleaved, a difference in the fluorescence may be detected, which indicates cleavage of an allele-specific probe comprising a pivotal nucleotide that is complementary to the polymorphic site. For non-limiting examples of 5′ nuclease assays, see, e.g., De La Vega et al. (2002) BioTechniques 32:S48-S54 (describing the TaqMan assay); Ranade et al. (2001) Genome Res. 11:1282-1268; and Shi, (2001) Clin. Chem. 47:164-172.

The term “a PCR assay employing molecular beacons” refers to an assay in which the polymerase drain reaction is used to amplify a region of a SNP locus comprising a polymorphic site. The reaction takes place in the presence of one or more allele-specific probes. When more than one allele-specific probe is used, the allele-specific probes may comprise different pivotal nucleotides. The allele-specific probes also comprise different fluorescent molecules. The allele-specific probes also comprise fluorescence quenching moieties. Allele-specific probes comprising a pivotal nucleotide that is not complementary to the polymorphic site will not substantially hybridize to the SNP locus during the annealing stage of PCR. Allele-specific probes comprising a pivotal nucleotide that is complementary to the polymorphic site will hybridize to the SNP locus during the annealing stage of PCR. When an allele-specific probe is not hybridized to the SNP locus, the quenching moiety is closer to the fluorescent molecule than when the probe is hybridized to the SNP locus. Thus, when allele-specific probes hybridize to the SNP locus, an increase in fluorescence occurs. Detection of an increase in fluorescence indicates which allele-specific probe has hybridized to the polymorphic site. For non-limiting examples of assays employing molecular beacons, see, e.g., Tyagi et al. (1998) Nature Biotech. 18:49-53; and Mhlanga et al. (2001) Methods 25:463-71.

The term “an assay employing flap endonuclease” refers to an assay in which a SNP locus is combined with one or more allele-specific probes and a second probe. In certain such embodiments, the allele-specific probe selectively hybridizes to a region comprising the polymorphic site and nucleotides that are 5′ of the polymorphic site. When more than one allele-specific probe is used, the allele-specific probes may comprise different pivotal nucleotides. The second probe selectively hybridizes to a region comprising the polymorphic site and nucleotides that are 3′ of the polymorphic site. When an allele-specific probe comprising a pivotal nucleotide that is complementary to the polymorphic site hybridizes adjacently to the second probe, a distinctive structure is formed. This structure is recognized and cleaved by a “flap” endonuclease, which results in the production of an increased fluorescent signal, in comparison to situations in which cleavage does not occur. When an allele-specific probe comprising a pivotal nucleotide that is not complementary to the polymorphic site hybridizes adjacently to the second probe, a distinctive structure is not substantially formed, so that the appropriate increase in fluorescent signal does not occur. For non-limiting examples of assays employing flap endonuclease, see, e.g., Hsu et al. (2001) Clin. Chem. 47:1373-1377 (describing the Invader® assay); Mein at al. (2000) Genome Res. 10:330-343; Ohnishi et al. (2001) J. Hum Gen. 48:471-477; and U.S. patent application Ser. No. 10/693,609, filed Oct. 23, 2003, corresponding to U.S. Patent Application Publication No. US 2004/0235006 A1.

The term “oligonucleotide ligation assay” refers to an assay in which a SNP locus is combined with one or more allele-specific probes and a second probe. When more than one allele-specific probe is used, the allele-specific probes may comprise different pivotal nucleotides. In certain embodiments, the pivotal nucleotide is located at the 5′ end of an allele-specific probe. In certain embodiments, the pivotal nucleotide is located at the 3′ end of an allele-specific probe. In certain embodiments, the pivotal nucleotide is located between the 5′ end and the 3′ end of the allele-specific probe. The allele-specific probe and the second probe hybridize immediately adjacent to each other at the SNP locus, such that the 5′ end of one of the probes is adjacent to the 3′ end of the other probe. Under ligation conditions, allele-specific probes comprising a pivotal nucleotide that is complementary to the polymorphic site become ligated to the second probes, resulting in ligation products. The ligation products are detected either directly or after one or more additional processes take place, such as an amplification reaction. Under ligation conditions, allele-specific probes comprising a pivotal nucleotide that is not complementary to the polymorphic site do not substantially ligate to the second probes. In certain embodiments of oligonucleotide ligation assays, the ligation product comprises a label. For non-limiting examples of oligonucleotide ligation assays, see, e.g., Grossman et al. (1994) Nuc. Acids Res. 22:4527-4534; U.S. patent application Ser. No. 09/584,905, filed May 30, 2000; U.S. patent application Ser. No. 10/011,993, filed Dec. 5, 2001, corresponding to U.S. Patent Application Publication No. US 2003/0119004 A1; Patent Cooperation Treaty Application No. PCT/US01/17329, filed May 30, 2001, corresponding to PCT International Publication No. WO 01/92579 A2; published Dec. 6, 2001; Patent Cooperation Treaty Application No. PCT/US97/45559, filed May 27, 1997; and U.S. Pat. No. 6,027,889, issued Feb. 22, 2000.

The term “label” refers to any molecule that can be detected. In certain embodiments, a label can be a moiety that produces a signal or that interacts with another moiety to produce a signal. In certain embodiments, a label can interact with another moiety to modify a signal of the other moiety. In certain embodiments, a label can bind to another moiety or complex that produces a signal or that interacts with another moiety to produce a signal. A complex encompasses more than one moiety associated by at least one covalent and/or at least one non-covalent interaction.

The term “amplification product” refers to the product of an amplification reaction including, but not limited to, primer extension, the polymerase chain reaction, RNA transcription, and the like. Thus, exemplary amplification products may comprise one or more products selected from primer extension products, PCR amplicons, RNA transcription products, and the like.

An “output” refers to a reading derived either directly or indirectly from an instrument that detects one or more labels.

The term “set of primers” refers to at least one primer that, under suitable conditions, specifically hybridizes to and amplifies a target sequence. In certain embodiments, a set of primers comprises at least two primers.

The term “STR-specific primer set” refers to at least two primers that are used for analyzing an STR locus.

In this application, a statement that one sequence is the same as or is complementary to another sequence encompasses situations where both of the sequences are completely the same or complementary to one another, and situations where only a portion of one of the sequences is the same as, or is complementary to, a portion or the entire other sequence. Here, the term “sequence” encompasses, but is not limited to, nucleic acid sequences, polynucleotides, oligonucleotides, probes, primers, primer-specific portions, and target-specific portions.

In this application, a statement that one sequence is complementary to another sequence encompasses situations in which the two sequences have mismatches. Here, the term “sequence” encompasses, but is not limited to, nucleic acid sequences, polynucleotides, oligonucleotides, probes, primers, primer-specific portions, and target-specific portions. Despite the mismatches, the two sequences should selectively hybridize to one another under appropriate conditions.

The term “selectively hybridize” means that, for particular identical sequences, a substantial portion of the particular identical sequences hybridize to a given desired sequence or sequences, and a substantial portion of the particular identical sequences do not hybridize to other undesired sequences. A “substantial portion of the particular identical sequences” in each instance refers to a portion of the total number of the particular identical sequences, and if does not refer to a portion of an individual particular identical sequence. In certain embodiments, “a substantial portion of the particular identical sequences” means at least 70% of the particular identical sequences. In certain embodiments, “a substantial portion of the particular identical sequences” means at least 80% of the particular identical sequences. In certain embodiments, “a substantial portion of the particular identical sequences” means at least 90% of the particular identical sequences. In certain embodiments, “a substantial portion of the particular identical sequences” means at least 95% of the particular identical sequences.

In certain embodiments, the number of mismatches that may be present may vary in view of the complexity of the composition. Thus, in certain embodiments, the more complex the composition, the more likely undesired sequences will hybridize. For example, in certain embodiments, with a given number of mismatches, a probe may more likely hybridize to undesired sequences in a composition with the entire genomic DNA than in a composition with fewer DNA sequences, when the same hybridization and wash conditions are employed for both compositions. Thus, that given number of mismatches may be appropriate for the composition with fewer DNA sequences, but fewer mismatches may be more optimal for the composition with the entire genomic DNA.

In certain embodiments, sequences are complementary if they have no more than 20% mismatched nucleotides. In certain embodiments, sequences are complementary if they have no more than 15% mismatched nucleotides. In certain embodiments, sequences are complementary if they have no more than 10% mismatched nucleotides. In certain embodiments, sequences are complementary if they have no more than 5% mismatched nucleotides.

In this application, a statement that one sequence hybridizes or binds to another sequence encompasses situations where the entirety of both of the sequences hybridize or bind to one another, and situations where only a portion of one or both of the sequences hybridizes or hinds to the entire other sequence or to a portion of the other sequence.

### B. CERTAIN EXEMPLARY EMBODIMENTS

In various embodiments, a method for genotyping a sample comprising nucleic acid is provided. In certain embodiments, the method comprises analyzing a plurality of STR foci and analyzing at least one SNP locus in the sample. In certain embodiments, the information obtained from analyzing a plurality of STR loci and analyzing at least one SNP locus may be used in various applications, for example, in genetic mapping, linkage analysis, clinical diagnostics, or identify testing. In certain embodiments, the information may be used to identify the source, or narrow down the possible sources, of the nucleic acid. In certain such embodiments, the information may be used, e.g., in forensic identification, paternity testing, DNA profiling, and related applications.

In certain embodiments, a sample comprising nucleic acid may be any source of biological material. In certain embodiments, a sample comprising nucleic acid may be biological material obtained, e.g., from a crime scene or from a site containing human or animal remains, such as an archeological site or a disaster site. In certain embodiments, nucleic acid is extracted from the biological material. See, e.g., Butler, Forensic DNA Typing, at pages 28-32. In certain embodiments, the biological material, including the nucleic acid, may be degraded or present in low amounts.

In certain embodiments, information obtained from analyzing at least one SNP locus is useful in combination with information from a plurality of STR loci in situations where information from STR loci alone fails to identify the source of the nucleic acid with a sufficient degree of confidence. In certain instances, such a situation may arise, for example, if the nucleic acid is degraded. In general, STRs occupy larger chromosomal regions than SNPs, which occur at single polymorphic nucleotides. Thus, in certain instances, if a nucleic acid is degraded, one may have a greater chance of success in identifying a SNP allele than in identifying an STR allele. Another situation may arise, for example, where a plurality of STR alleles are identified in a sample, but the STR profile thus obtained fails to match a known STR profile. For example, a sample from a crime scene may yield an STR profile that fails to match any STR profile in a database of known offenders. However, in certain embodiments, identification of certain SNP alleles present in the sample may provide information on the phenotype of the perpetrator of the crime, e.g., eye color, hair color, ethnicity, and the like. In certain embodiments, this information may be used to help narrow down potential suspects from whom biological samples may be obtained. In certain embodiments, STR profiles from those biological samples may then be compared with the STR profile of the crime scene sample.

### 1. Certain Embodiments of STR Analysis

In certain embodiments, e.g., in certain identity testing methods, a plurality of STR loci are selected based on certain criteria that increase the likelihood of accurate identification. In certain embodiments, the STR loci selected for analysis are highly polymorphic. In certain embodiments, STR loci from different chromosomal locations are chosen to reduce the chance of closely linked STR loci. In certain embodiments, STR loci are chosen that have a low mutation rate. In certain embodiments, STR loci are chosen that typically have higher rates of accurate amplification by PCR. In certain embodiments, STR loci comprising tetranucleotide repeats are chosen. In certain embodiments, the STR loci are selected to fall in a size range of about 50 to about 300 base pairs.

In certain embodiments, a plurality of STR loci to be analyzed are selected from “STRSase,” the STR database compiled and maintained by the National Institute of Standards and Technology (NIST). See, e.g., Ruitberg et al. (2001) “STRBase: a short tandem repeat DNA database for the human identity testing community,” Nuc. Acids Res. 29:320-322; and world wide website cstl.nist.gov/biotech/strbase/.

In certain embodiments, a plurality of STR loci comprise one or more autosomal STR loci. In certain embodiments, a plurality of STR loci comprise one or more STR loci from the X chromosome. In certain embodiments, a plurality of STR loci comprise one or more STR loci from the Y chromosome. Certain exemplary STR loci from the autosomes, X-chromosome, and Y-chromosome are known to those skilled in the art. See, e.g., Butler, Forensic DNA Typing, supra, at pages 64, 74, and 121; Ruitberg et al., supra; and world wide website cstl.nist.gov/biotech/strbase/. In certain embodiments, the chromosomal locations of STR loci may be determined empirically.

In certain embodiments, a plurality of STR loci comprise any one or more of the thirteen CODIS STR loci. In certain embodiments, a plurality of STR loci comprise all thirteen CODIS STR loci. (See, e.g., the AmpFLSTR Identifier® PCR amplification kit from Applied Biosystems, Foster City, Calif.) In certain embodiments, a plurality of STR loci comprise the CODIS STR loci of vWA, FGA, D3S1368, D5S818, D7S820, D13S317, D8S1179, D18S51, and D21S11. (See, e.g., the AmpFLSTR Profiler Plus® PCR amplification kit from Applied Biosystems.) In certain embodiments, a plurality of STR loci comprise the CODIS STR loci of TH01, TPOX, CSF1PO, vWA, FGA, D3S1358, D5S818, D7S820, and D13S317. (See, e.g., the AmpFLSTR Profiler® PCR amplification kit from Applied Biosystems.) In certain embodiments, a plurality of STR loci comprise the CODIS STR loci of CSF1PO, D16S539, TH01, TPOX, D3S1358, and D7S820. (See, e.g., the AmpFLSTR COfiler® PCR amplification kit from Applied Biosystems.) In certain embodiments, a plurality of STR loci comprise the CODIS STR loci of D3S1358, vWA, and FGA. (See, e.g., the AmpFLSTR Blue® PCR amplification kit from Applied Biosystems.) In certain embodiments, a plurality of STR loci comprise the CODIS STR loci of TH01, TPOX, and CSF1PO. (See, e.g., the AmpFLSTR Green™ I PCR amplification kit from Applied Biosystems.) In certain embodiments, a plurality of STR foci comprise the CODIS STR loci of D21S11, FGA, TH01, vWA, D8S1179, and D18S51. (See, e.g., the AmpFLSTR SGM Plus® PCR amplification kit from Applied Biosystems.)

In certain embodiments, a plurality of STR loci comprise one or more non-CODIS STR loci. Exemplary non-CODIS STR loci include, but are not limited to, ARA, APOAI1, ACPP, ACTBP2, CD4, CYAR04, CYP19, F13A01, F13B, FABP, FES/FPS, FOLP23, GABARB15, HPRTB, LPL, MBP, Penta D, Penta E, PLA2A1, RENA4, SE33, STRX1, UGB, D1S103, D1S1171, D1S1856, D2S410, D2S438, D2S1242, D2S1338, D3S1349, D3S1352, D3S1359, D3S1744, D5S373, D5S815, D6S368, D8S477, D8S502, D8S985, D7S460, D7S8Q9, D7S1517, D7S1520, D8S320, D8S323, D8S344, D8S347, D8S839, D8S1179, D9S52, D9S302, D10S89, D10S2325, D11S488, D11S554, D12S87, D12S391, D12S1090, D13S308, D18S537, D17S978, D18S535, D18S849, D19S433, D20S85, D20S181, D22S883, DXS8807, DXYS156, DYS19, DYS385, DYS388, DYS389 I, DYS389 II, DYS390, DYS 391, DYS392, DYS393, YCAIII, DYS434, DYS435, DYS438, DYS437, DYS438, DYS439, Y-GATA-A4, Y-GATA-A7.1, Y-GATA-A7.2, Y-GATA-A8, Y-GATA-A10, Y-GATA-C4, and Y-GATA-H4. See, e.g., world wide website cstl.nist.gov/biotech/strbase/.

In certain embodiments, a plurality of STR loci comprise a combination of one or more CODIS STR loci and one or more STR loci that are non-CODIS STR loci. In certain embodiments, a plurality of STR loci comprise TH01, TPOX, CSF1PO, vWA, D3S1358, D7S820, D13S317, D16S539, D8S1179, D18S51, D21S11, D2S1338, and D19S433. In certain embodiments, a plurality of STR loci comprise TM01, TPOX, CSF1PO, vWA, FGA, D3S1358, D5S818, D7S820, D13S317, D16S539, D8S1179, D18S51, D21S11, D2S1338, and D19S433. (See, e.g., the AmpFLSTR Identifier® PCR amplification kit from Applied Biosystems.) In certain embodiments, a plurality of STR loci comprise TH01, vWA, FGA, D3S1358, D16S539, D8S1179, D18S51, D21S11, D2S1338, and D19S433. (See, e.g., the AmpFLSTR SGM Plus® PCR amplication kit from Applied Biosystems.) In certain embodiments, a plurality of STR loci comprise TH01, vWA, FGA, D3S1358, D16S539, D8S1179, D18S51, D21S11, D2S1338, D19S433, D22S684, D10S516, D14S306, and D1S518. (See, e.g., the AmpFLSTR TGM® PCR amplification kit from Applied Biosystems.) In certain embodiments, a plurality of STR foci comprise TK01, vWA, FGA, D2S1338, D3S1358, D8S1179, D16S539, D18S51, D19S433, D21S11, and SE33. (See, e.g., the AmpFLSTR SEfiler® PCR amplification kit from Applied Biosystems.)

In certain embodiments, the marker amelogenin is analyzed along with a plurality of STR loci in order to identify the gender of the source of the nucleic acid. Those skilled in the art are familiar with the analysis of amelogenin.

In certain embodiments, the STR alleles present at a plurality of STR loci are identified. In certain such embodiments, an STR allele is identified by determining the size of a region comprising the repeating units of an STR locus. In certain such embodiments, an STR allele is identified by determining the number of repeating units at an STR locus.

In certain embodiments, a plurality of STR loci are amplified by the polymerase chain reaction (PCR) using STR-specific primer sets. An STR-specific primer set comprises at least two primers for amplifying a target STR locus. The primers of an STR-specific primer set hybridize to regions of the target STR locus that flank the repeating units. In other words, at least one primer of an STR-specific primer set hybridizes to a region that is located 5′ of the repeating units, and at least one primer of an STR-specific primer set hybridizes to a region that is located 3′ of the repeating units. In certain embodiments, one skilled in the art can routinely select primers for a plurality of STR-specific primer sets using commercially available primer design software packages, including but not limited to, Primer Express (Applied Biosystems, Foster City, Calif.). In certain embodiments, a plurality of STR-specific primer sets are available in commercially available kits, for example, the AmpFLSTR Identifiler® PCR amplification kit (Applied Biosystems, Foster City, Calif.), which contains primer sets that amplify the TH01, TPOX, CSF1PO, vWA, FGA, D3S1358, D5S818, D7S820, D13S317, D16S539, D8S1179, D18S51, D21S11, D2S1338, and D19S433 loci.

In certain embodiments, at least one primer of an STR-specific primer set comprises a label. In certain embodiments, at least one primer of an STR-specific primer set comprises a mobility modifier. In certain embodiments, at least one primer of an STR-specific primer set comprises a portion that does not hybridize to the target STR locus. In certain embodiments, the portion that does not hybridize to the target STR locus is located at the 5′ end of the at least one primer. In certain embodiments, the portion that does not hybridize to the target STR locus comprises a sequence that is the same as the sequence of a “universal” primer. Those skilled in the art are familiar with certain universal primers and their use in certain amplification reactions. See, e.g., Lin et al. (1996) Proc. Nat'l Acad. Sci. USA 93:2582-2587. In certain such embodiments, the universal primer may then be used to amplify the amplification products generated by one or more different STR-specific primer sets. In certain embodiments, a universal primer comprises a label. In certain embodiments, a universal primer comprises a mobility modifier.

In certain embodiments, wherein at least one primer of an STR-specific primer set comprises a portion that does not hybridize to the target STR locus, the portion that does not hybridize to the target STR locus is used to vary the size of the amplification product generated by the STR-specific primer set. For example, in certain embodiments, increasing the length of the portion that does not hybridise to the target STR locus increases the size of the amplification produces) generated by an STR-specific primer set. In certain embodiments, the portion that does not hybridize to the target STR locus hybridizes to a nucleic acid attached to a mobility modifier, which is used to differentiate amplification products by their mobilities. A mobility modifier is any moiety that alters the migration of a polynucleotide in a mobility-dependent analysis technique, such as electrophoresis. Certain mobility modifiers are described, e.g., in U.S. Pat. No. 6,395,486 B1, issued May 28, 2002; and Grossman et al. (1994) Nuc. Acids Res. 22:4527-4534.

In certain embodiments, a given STR-specific primer set may generate one or more amplification products, depending on whether the nucleic acid being genotyped is homozygous or heterozygous at the target STR locus. In certain embodiments, the size of the one or more amplification products is a function of the number of repeating units at the target STR locus. Therefore, in such embodiments, the size of the one or more amplification products indicates the identify of the STR allele or alleles at the target STR locus. For example, in certain embodiments, the generation of only one amplification product having a size that corresponds to nine repeating units indicates that the target STR locus is homozygous for that particular nine-unit STR allele. In certain embodiments, the generation of two amplification products of different sizes corresponding to, for example, nine repeating units and eight repeating units, respectively, indicates heterozygosity at the target STR locus for those two particular STR alleles.

In certain embodiments, for example, when the sample to be genotyped is highly degraded, the primers of an STR-specific primer set may closely flank the repeating units, thus increasing the likelihood of obtaining an amplification product.

In certain embodiments, a plurality of STR loci are amplified in the same reaction mixture using a plurality of STR-specific primer sets. See, e.g., U.S. Pat. No. 6,221,598 B1. In certain such embodiments, the plurality of STR loci and the plurality of STR-specific primer sets are selected so that there is minimal overlap among the sizes of the amplification products generated from different STR loci, particularly where those amplification products comprise the same label. In certain embodiments, commercially available kits are used to amplify a plurality of STR loci in the same reaction mixture. (See, e.g., the AmpFLSTR® series of PCR amplification kits from Applied Biosystems, Foster City, Calif.) In certain embodiments, the amplification products generated by the STR-specific primer sets may be further amplified in the same reaction mixture using one or more universal primers. In certain embodiments, amplification products from different STR loci that overlap in size are differentiated using different mobility modifiers.

In certain embodiments, the sizes of a plurality of amplification products are determined. In certain embodiments, a plurality of amplification products in a single reaction mixture are subjected to an analytical technique that separates the amplification products based on their sizes. In certain such embodiments, the analytical technique separates the amplification products based on their eleetrophorefic mobilities. Those skilled in the art are familiar with certain of such techniques. For a review, see, e.g., Butler, Forensic DNA Typing, supra, at pages 135-146.

In certain embodiments, the sizes of a plurality of amplification products are determined using slab gel electrophoresis. In certain such embodiments, polyacrylamide gel electrophoresis under either native or denaturing conditions is used. In certain embodiments, the amplification products comprise one or more labels, which are incorporated into the amplification products during or after the PCR. In certain such embodiments, the labels are fluorescent labels, which are detected by a laser scanner. See e.g., Butler, Forensic DNA Typing, supra, at pages 138-140.

In certain embodiments, the sizes of a plurality of amplification products are determined using capillary electrophoresis (CE). Those skilled in the art are familiar with certain CE techniques. For a review, see, e.g., Butler, Forensic DNA Typing, supra, at pages 140-143. In certain such embodiments, the amplification products comprise one or more labels that are incorporated into the amplification products during or after the PCR. In certain such embodiments, the labels are fluorescent labels, which are detected by a laser during separation of the amplification products by CE. In certain embodiments, CE is carried out using an Applied Biosystems 310 or 3100 Capillary DNA Sequencer/Genotyper (Applied Biosystems, Foster City, Calif.). In certain embodiments, multiple capillary channels are present on an array, enabling processing of multiple samples in parallel. See, e.g., Mansfield et al. (1996) Genome Res. 6:893-903; and Medintz et al. (2001) Clin. Chem. 47:1614-1621. In certain embodiments, microchip gel electrophoresis is used. See, e.g., Schmalzing et al. (1997) Proc. Nat'l Acad. Sci. USA 94:10273-78.

In certain embodiments, amplification products comprise one or more fluorescent labels that are suitable for detection in CE analysis. Certain exemplary fluorescent labels include, but are not limited to, 6-FAM™ (6-carboxy fluorescein), VIC®, NED®, PET®, LIZ®, 5-FAM™ (5-carboxy fluorescein), JOE™ (6-carboxy-2′,7′-dimethoxy-4′,5′-dichlorofluorescein), and ROX™ (6-carboxy-X-rhodamine) (Applied Biosystems, Foster City, Calif.); fluorescein; TAMRA™ (N,N,N′,N′-tetramethyl-6-carboxyrhodamine); TET (4,7,2′,7′-tetrachloro-6-carboxyfluroescein); HEX (4,7,2′,4′,5′,7′-hexachloro-6-carboxyfluorescein); and SYBR green (Molecular Probes, Eugene, Oreg.).

In certain embodiments, one skilled in the art can select appropriate labels based on the sizes of the amplification products and on whether the amplification products are present in the same reaction mixture. For example, in certain embodiments, if a reaction mixture comprises amplification products generated from different STR loci, and those amplification products overlap in size, then those amplification products may comprise different fluorescent labels so that they may be distinguished from one other. In certain such embodiments, the reaction mixture may be analyzed in a single lane of a slab gel or in a single capillary channel of a CE apparatus. In certain embodiments, if a reaction mixture comprises amplification products generated from different STR loci, and those amplification products do not overlap in size, then those amplification products may comprise the same label, in certain such embodiments, the reaction mixture is analyzed in a single lane of a slab gel or in a single capillary channel of a CE apparatus. In certain such embodiments, the amplification products are distinguishable from one another because they migrate to distinct regions within the slab gel or because they migrate at non-overlapping rates through the capillary channel.

In certain embodiments, the size of one or more amplification products is determined using mass spectrometry, including MALDI-TOF. Certain such embodiments are described, for example, in Butler et al. (1998) Int. J. Legal. Med. 112:45-49. In certain such embodiments, a plurality of STR loci are amplified using primer sets that generate amplification products of about 150 base pairs or less.

### 2. Certain Embodiments of SNP Analysis

In certain embodiments, at least one SNP locus is analyzed in a sample. Certain exemplary SNP loci include, but are not limited to, those compiled by “The SNP Consortium” (TSC). See, e.g., Thorisson et al. (2003) Nuc. Acids. Res. 31:124-127; see also world wide website snp.cshl.org/. Certain exemplary SNP loci include, but are not limited to, loci comprising the following SNPs (listed by TSC identification number): TSC0252540, TSC1342445, TSC0421788, TSC0478751, TSC0320708, TSC0155410, TSC0154197, TSC0883201, TSC0739545, TSC0131214, TSC0158245, TSC0709018, and TSC0078283. See, e.g., world wide website cstl.nist.gov/div831/strbase/SNP.htm.

In certain embodiments, the analysis of at least one SNP locus provides information on the phenotype of the source of the sample being genotyped. In certain embodiments, the analysis of at least one SNP locus provides information on the ancestral origin (ethnicity) of the source of the sample being genotyped. For certain exemplary SNP alleles that are associated with human ancestral origin, see, e.g., Frudakis et al. (2003) J. Forensic Sci. 48(4):1-8 and Appendix I. Such exemplary SNP alleles are found in human genes including, but not limited to, OCA2, TYRP1, TYR, CYP2D6, CYP2C9, CYP3A4, MC1R/MSHR, CYP1A1, AHR, HMGCR, and FDPS.

In certain embodiments, the information on phenotype provides information on gender, hair color, eye color, and/or skin color. For certain exemplary SNP alleles that are associated with eye color, see, e.g., Frudakis et al. (2003) Genetics 165:2071-2083. Such exemplary SNP alleles are found in human genes including, but not limited to, OCA2, TYRP1, AIM, MYO5A, DCT, CYP2C8, CYP2C9, CYP1B1, and MAOA. For certain exemplary SNP alleles that are associated with skin color, see, e.g., Frudakis et al. (2003) Genetics 165:2071-2083; Frudakis et al (2003) J. Forensic Sci. 48(4):1-8 and Appendix I; and Shriver et al. (2003) Hum. Genet. 112:387-399. Such exemplary SNP alleles are found in human genes including, but not limited to, OCA2, TYRP1, TYR, MC1R (MSHR), AP3B1, ASIP, DCT, SLIV, MYO5A, POMC, AIM, AP3D1, and RAB. For certain exemplary SNP alleles that are associated with hair color, see, e.g., Box et al. (1997) Hum. Mol. Gen. 6:1891-1897; Frudakis et al. (2003) Genetics 165:2071-2083; and Grimes et al. (2001) Forensic. Sci. Int'l 122:124-129. Such exemplary SNP alleles are found in human genes including, but not limited to, MC1R (MSHR).

In certain embodiments, the at least one SNP locus is autosomal. In certain embodiments, the at least one SNP locus is on the Y-chromosome. Certain exemplary Y-chromosome SNPs are described, e.g., in Underhill et al. (2000) Nature Genetics 26:358-361. In certain embodiments, for example, in which the sample to foe genotyped is degraded, the at least one SNP locus is mitochondrial. Certain exemplary mitochondrial SNPs are described, e.g., in Vallone et al. (2004) Int. J. Legal Med., Feb. 4, 2004 (e-publication in advance of printed publication).

In various embodiments, any of a number of methods can be used to analyze a SNP locus. Those skilled in the ad are familiar with certain of such methods, which are reviewed, for example, in Syvänen (2001) Nat. Rev. Genet. 2:930-42; Kwok (2001) Annu. Rev. Hum. Genet. 2:235-58; and Shi (2001) Clin. Chem. 47:164-72. In certain embodiments, a SNP locus is analyzed using a method selected from an extension assay, an allele-specific oligonucleotide hybridization assay, a 5′ nuclease assay, a PCR assay employing molecular beacons, an assay employing flap endonuclease, or an oligonucleotide ligation assay.

Certain exemplary extension assays are known to those skilled in the art. Exemplary extension assays include, but are not limited to, allele-specific primer extension assays, allele-specific PCR, allele-specific nucleotide incorporation assays, and single base extension assays. In certain embodiments, in any of the above extension assays, one or more SNP alleles may be identified by the presence of an extension product. In certain embodiments, an extension product is detected by the detection of a label. In certain such embodiments, the particular label that is detected indicates which one or more of the possible SNP alleles are present in the sample.

In certain embodiments, at least one SNP locus is analyzed using an allele-specific primer extension assay. In certain embodiments, at least a portion of the sample to be genotyped is combined with at least one allele-specific primer and a polymerase. In certain embodiments, the pivotal nucleotide of the at least one allele-specific primer is located at the 3′ end of the allele-specific primer. When an allele-specific primer comprises a pivotal nucleotide that is complementary to the nucleotide at a polymorphic site, a polymerase is capable of adding nucleotides to the 3′ end of the allele-specific primer, thus resulting in an extension product. In certain embodiments, an extension product is detected by the detection of a label. In certain embodiments, the particular label that is detected indicates which allele-specific primer was extended, and therefore, which pivotal nucleotide is complementary to the nucleotide at the polymorphic site. In this manner, a SNP allele may be identified.

In certain embodiments, an allele-specific primer comprises a label. In certain embodiments, an allele-specific primer comprises a mobility modifier. In certain embodiments, the at least one allele-specific primer comprises a portion that does not hybridize to the SNP locus. In certain embodiments, the portion that does not hybridize to the SNP locus is located at the 5′ end of the at least one allele-specific primer. In certain embodiments, the portion that does not hybridize to the SNP locus comprises a sequence that is the same as the sequence of a universal primer. Those skilled in the art are familiar with certain universal primers and their use in certain amplification reactions. See, e.g., Lin et al. (1996) Proc. Nat'l Acad. Sci. USA 93:2582-2587. In certain such embodiments, the universal primer may then used to amplify the amplification products generated by the at least one allele-specific primer. In certain embodiments, a universal primer comprises a label. In certain embodiments, a universal primer comprises a mobility modifier.

In certain embodiments, wherein the at least one allele-specific primer comprises a portion that does not hybridize to the SNP locus, the portion that does not hybridize to the SNP locus is used to vary the size of the amplification product generated by the at least one allele-specific primer. For example, in certain embodiments, increasing the length of the portion that does not hybridize to the SNP locus increases the size of the amplification product generated by the at least one allele-specific primer. In certain embodiments, the portion that does not hybridize to the SNP locus hybridizes to a nucleic acid attached to a mobility modifier, which is used to differentiate amplification products by their mobilities. A mobility modifier is any moiety that alters the migration of a polynucleotide in a mobility-dependent analysis technique, such as electrophoresis. Certain mobility modifiers are described, e.g., in U.S. Pat. No. 6,395,486 B1; and Grossman et al. (1994) Nuc. Acids Res. 22:4527-4534.

In certain embodiments, more than one allele-specific primer is used to analyze a single SNP locus. In certain such embodiments, the allele-specific primers comprise different pivotal nucleotides. In certain embodiments, allele-specific primers comprising different pivotal nucleotides comprise different labels. In certain embodiments, allele-specific primers comprising different pivotal nucleotides comprise different mobility modifiers. In certain embodiments, allele-specific primers comprising different pivotal nucleotides further comprise portions that do not hybridize to the SNP locus. In certain embodiments, those portions are of different lengths. In certain embodiments, those portions comprise different sequences. In certain embodiments, those portions hybridize to nucleic acids attached to different mobility modifiers.

In certain embodiments, an allele-specific primer extension assay is allele-specific PCR. In certain such embodiments, at least a portion of the sample to be genotyped is combined with at least one first set of primers, wherein the at least one first set of primers comprises at least one allele-specific primer and a second primer. In certain embodiments, the at least one allele-specific primer and, optionally, the second primer, comprise portions that do not hybridize to the SNP locus. In certain embodiments, those portions are located at the 5′ ends of the primers. In certain embodiments, those portions comprise sequences that are the same as the sequence of one or more universal primers. The one or more universal primers may then be used to amplify the amplification products generated by the at least one first set of primers. In certain embodiments, at least one of the one or more universal primers comprises a label. In certain embodiments, at least one of the one or more universal primers comprises a mobility modifier. Certain methods using allele-specific PCR and universal primers to identify the SNP alleles at multiple SNP foci are known in the art. See, e.g., Myakishev et al. (2001) Genome Res. 11:163-169; Hawkins et al. (2002) Hum. Mut. 19:543-553; and Bengra et al. (2002) Clin. Chem. 48:2131-2140; and PCT publication WO 02/103045 A2.

In certain embodiments, extension product(s) resulting from an extension assay are subjected to an analytical technique that separates the extension product(s) based on their sizes. In certain such embodiments, the analytical technique is any of the techniques described above for analysis of STR loci.

In certain embodiments, at least one SNP locus is analyzed by combining at least a portion of the sample to be genotyped with at least one allele-specific probe and detecting hybridization of the at least one allele-specific probe to the SNP locus. In certain such embodiments, hybridization is detected when the pivotal nucleotide of the allele-specific oligonucleotide is complementary to the nucleotide at the polymorphic site of the SNP locus. In certain such embodiments, hybridization is detected using an allele-specific hybridization assay, a 5′ nuclease assay, an assay employing molecular beacons, an assay employing flap endonuclease, or an oligonucleotide ligation assay. In certain embodiments, hybridization is detected based on the detection of a label.

In certain embodiments, at least one SNP locus is analyzed using an oligonucleotide ligation assay. In certain such embodiments, at least a portion of a sample to be genotyped is combined with at least one first set of probes. In certain such embodiments, the at least one first set of probes comprises one or more allele-specific probes and a second probe for each SNP locus that is to be analyzed. In certain embodiments, at least one probe from the first set of probes comprises a label. In certain embodiments, at least one probe from the first set of probes comprises a mobility modifier. In certain embodiments, the pivotal nucleotide of the one or more allele-specific probes is located at the 3′ end of the allele-specific probes. In certain such embodiments, the one or more allele-specific probes and the second probe hybridize to the SNP locus, such that the second probe hybridizes to the SNP locus at a nucleotide sequence that is immediately 5′ of the nucleotide sequence to which the one or more allele-specific probes hybridize. When the pivotal nucleotide of an allele-specific probe is complementary to the nucleotide at the polymorphic site, the 3′ end of the allele-specific probe becomes ligated to the 5′ end of the second probe under appropriate conditions, resulting in a ligation product. In certain embodiments, the ligation product is detected by the detection of a label. In certain such embodiments, the particular label that is detected indicates which allele-specific probe was ligated to the second probe, and therefore, which pivotal nucleotide is complementary to the nucleotide at the polymorphic site. In this manner, a SNP allele may be identified.

In certain embodiments, at least one probe from the first set of probes comprises a portion that does not hybridize to the SNP locus. In certain embodiments, the portion that does not hybridize to the SNP locus is used to vary the size of the ligation product. For example, in certain embodiments, increasing the length of a portion that does not hybridize to the SNP locus increases the size of the ligation product. In certain embodiments, a portion that does not hybridize to the SNP locus hybridizes to a nucleic acid attached to a mobility modifier, which is used to differentiate ligation products (or amplified ligation products) by their mobilities. A mobility modifier is any moiety that alters the migration of a polynucleotide in a mobility-dependent analysis technique, such as electrophoresis. Certain mobility modifiers are described, e.g., in U.S. Pat. No. 6,395,486 B1, issued May 28, 2002; and Grossman et at. (1994) Nuc. Acids Res. 22:4527-4534.

In certain embodiments, portions that do not hybridize to the SNP locus are located at the 5′ ends of the one or more allele-specific probes and the 3′ end of the second probe. In certain embodiments, those portions comprise sequences that are the same as or complementary to one or more universal primers. In certain such embodiments, the ligation product produced by the ligation of an allele-specific probe to a second probe is amplified using one or more universal primers. In certain embodiments, the ligation products from different SNP loci may be amplified using a common set of universal primers. In certain embodiments, one or more universal primers comprise a label. In certain embodiments, one or more universal primers comprise a mobility modifier.

In certain embodiments, more than one allele-specific probe is used to analyze a single SNP locus in an oligonucleotide ligation assay, in certain such embodiments, the allele-specific probes comprise different pivotal nucleotides. In certain embodiments, allele-specific probes comprising different pivotal nucleotides comprise different labels. In certain embodiments, allele-specific probes comprising different pivotal nucleotides comprise different mobility modifiers, in certain embodiments, allele-specific probes comprising different pivotal nucleotides further comprise portions that do not hybridize to the SNP locus. In certain embodiments, these portions comprise different sequences. In certain embodiments, these portions are of different lengths. In certain embodiments, these portions hybridize to nucleic acids attached to different mobility modifiers.

In certain embodiments, one or more nucleic acids from an oligonucleotide ligation assay reaction mixture are subjected to an analytical technique that separates the nucleic acids based on their sizes. In certain such embodiments, the analytical technique is any of the techniques described above for analysis of STR loci.

### 3. Combined STR and SNP Analysis

In certain embodiments, a sample comprising nucleic acid is genotyped by analyzing a plurality of STR loci and at least one SNP locus in the sample. In certain embodiments, a portion of the sample may be subjected to any of the above methods for analyzing a plurality of STR loci, and another portion of the sample may be subjected to any of the above methods for analyzing at least one SNP locus.

In certain embodiments, a plurality of STR loci and at least one SNP locus are analyzed in the same reaction mixture. In certain such embodiments, the plurality of STR loci and the at least one SNP locus are amplified in the same reaction mixture. In certain such embodiments, the at least one SNP locus is amplified by an extension assay. In certain such embodiments, the extension assay is an allele-specific primer extension assay. In certain such embodiments, the allele-specific primer extension assay is allele-specific PCR. In certain embodiments, the reaction mixture is subjected to an analytical technique that separates the STR and SNP amplification products based on their sizes. In certain such embodiments, the analytical technique is any of the techniques described above for analysis of STR loci.

In certain embodiments in which the plurality of STR loci and the at least one SNP locus are amplified in the same reaction mixture, there is minimal or no overlap among the sizes of the STR and/or SNP amplification products that comprise the same label. In certain embodiments in which the plurality of STR loci and the at least one SNP locus are amplified in the same reaction mixture, STR and/or SNP amplification products that overlap In size comprise different labels. In this manner, amplification products may be distinguished from one another.

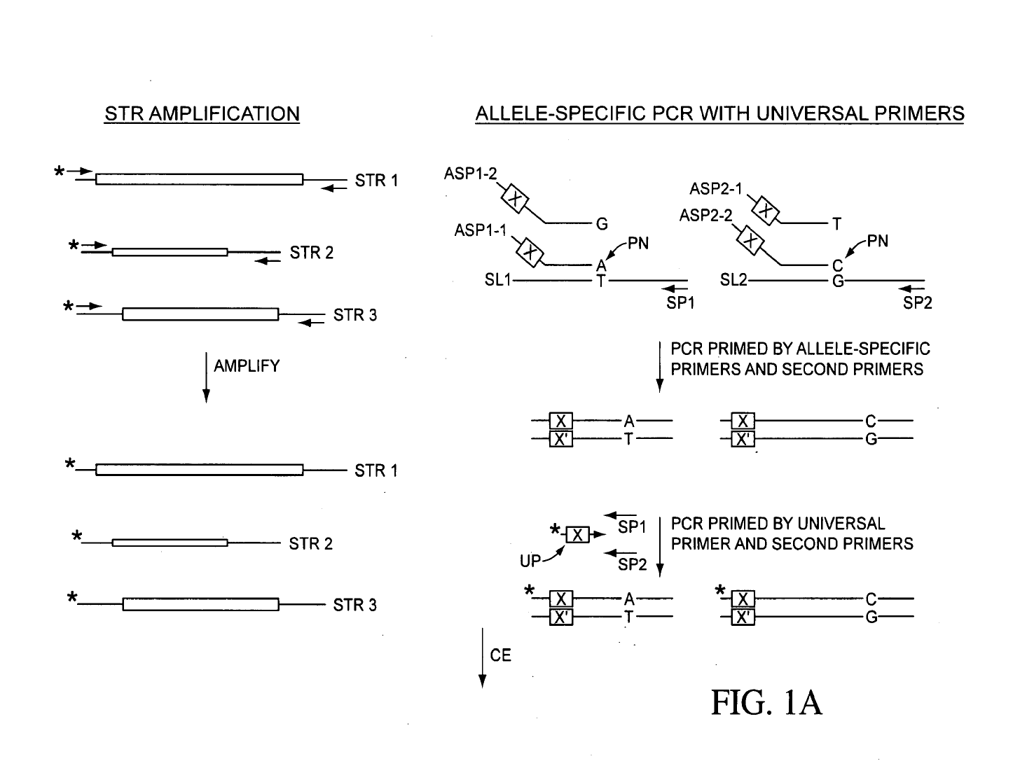

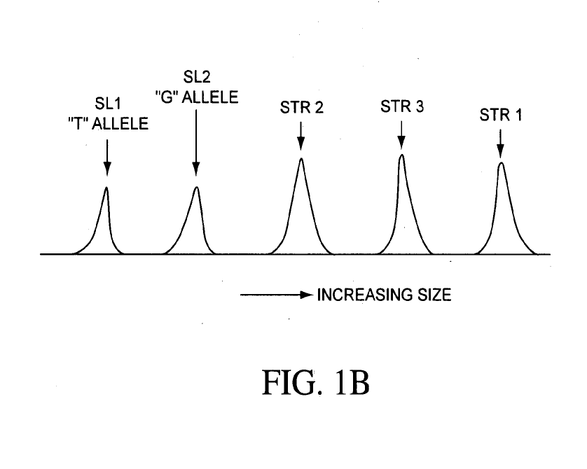

For example, in certain embodiments shown in FIG. 1, primers (→ and ←) from three STR-specific primer sets are used to amplify three different STR loci (STR1, STR2, and STR3) in a reaction mixture. (Only one strand of each STR locus is shown.) One primer from each STR-specific primer set comprises a label ( [symbol] ) that becomes incorporated into its corresponding amplification product. In the embodiments shown in FIG. 1, two bialleleic SNP loci (SL1 and SL2) are analyzed in the same reaction mixture using two first sets of primers. (Only one strand of each SNP locus is shown.) One of the first sets of primers comprises two allele-specific primers (ASR1-1 and ASP1-2) and a second primer (SP1). The other first set of primers comprises two allele-specific primers (ASP2-1 and ASP2-2) and a second primer (SP2). ASP1-1 comprises a pivotal nucleotide (PN) that is complementary to the nucleotide at the polymorphic site of SL1. ASP2-2 comprises a pivotal nucleotide (PN) that is complementary to the nucleotide at the polymorphic site of SL2. Thus, ASP1-1 and ASP2-2 are capable of generating amplification products.

In the embodiments shown in FIG. 1, the allele-specific primers comprise 5′ portions that do not hybridize to SL1 or SL2. In FIG. 1, the 5′ portions of two different allele-specific primers of a given first set of primers are of different lengths. Thus, amplification products comprising different SNP alleles corresponding to the same SNP locus can be distinguished based on size. The 5′ portions of the allele-specific primers also comprise a region ( [symbol] ) that is identical in sequence among the allele-specific primers. Region [symbol] comprises the same sequence as a universal primer (UP), which comprises a label ( [symbol] ). In the embodiments shown in FIG. 1, the second primers (SP1 and SP2) are positioned such that the size of the amplification products from SL1 and SL2 do not overlap with one another or with the size of any of the STR amplification products. In certain embodiments, to avoid overlap in size among the amplification products from SL1 and SL2, allele-specific probes (ASP1-1, ASP1-2, ASP2-1, and ASP2-2) each comprise 5′ portions of differing length.

In the embodiments shown in FIG. 1, the reaction mixture is subjected to PCR. The STR loci are amplified by the STR-specific primer sets. (Only one strand of each STR amplification product is shown.) Additionally, ASP1-1 and SP1 amplify SL1, and the resulting amplification product is amplified by UP and SP1. ASP2-2 and SP2 amplify SL2, and the resulting amplification product is amplified by UP and SP2. In the embodiments shown in FIG. 1, at least a portion of the reaction mixture is then subjected to CE in a single capillary channel. In the embodiments shown in FIG. 1, the amplification products are detected by their labels. In the embodiments shown in FIG. 1, the detection is displayed in a single output, represented schematically in FIG. 1B. The rate at which the STR amplification products migrate through the channel is a function of their size, which identifies the STR allele(s) at each STR locus. The rate at which the SNP amplification products migrate through the channel is also a function of their size, which identifies the SNP alleles present at the SL1 and SL2 loci.

In certain embodiments, the analyzing a plurality of STR loci and the analyzing at least one SNP locus comprise processes that occur in separate reaction mixtures, in certain such embodiments, the analyzing further comprises combining the separate reaction mixtures and detecting the STR and SNP alleles in the combined reaction mixture. For example, in certain embodiments, a portion of the sample to be genotyped is combined in a first reaction mixture with a plurality of STR-specific primer sets. The first reaction mixture is subjected to amplification. Another portion of the sample to be genotyped is combined in a second reaction mixture with one or more primers or probes that selectively hybridize to a SNP locus. The second reaction mixture is then subjected to hybridization and, optionally, amplification steps of an assay for analyzing SNPs. Such assays include, but are not limited to, extension assays and oligonucleotide ligation assays. The first and second reaction mixtures are then combined to form a combined reaction mixture. In certain embodiments, the combined reaction mixture is subjected to an analytical technique that separates and/or detects the nucleic acids therein. In certain such embodiments, the analytical technique is any of the techniques described above for analysis of STR loci.

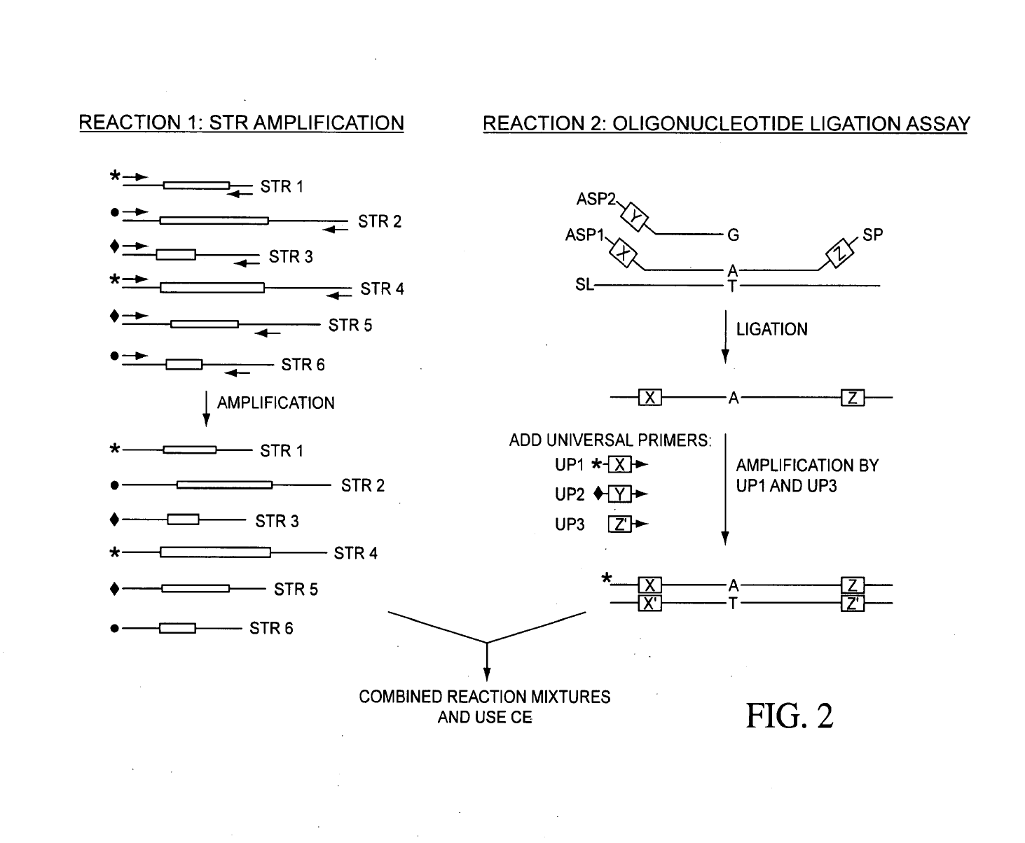

For example, in certain embodiments shown in FIG. 2, a portion of a sample comprising nucleic acid is amplified in a single reaction mixture using six different STR-specific primer sets. The STR-specific primer sets comprise primers (→ and ←) that amplify six different STR loci (STR1, STR2, STR3, STR4, STR5, and STR6). One primer from each STR-specific primer set comprises one of three different colored labels: ( [symbol] , ♦, or [symbol] ). The labels become incorporated into the amplification products of the STR-specific primer sets.

In the embodiments shown in FIG. 2, another portion of the same sample is subjected to an oligonucleotide ligation and PCR assay in a separate reaction mixture. The portion is combined with two allele-specific probes (ASP1 and ASP2) and a second probe (SP) that hybridize to a bialleleic SNP locus (SL). The allele specific probes comprise 5′ portions that do not hybridize to the SNP locus. The 5′ portion of ASP1 comprises a sequence ( [symbol] ) that is the same as the sequence of a first universal primer (UP1). The 5′ portion of ASP2 comprises a sequence ( [symbol] ) that is the same as the sequence of a second universal primer (UP2). The 3′ portion of SP1 comprises a sequence ( [symbol] ) that is complementary to a third universal primer (UP3, having sequence [symbol] ). Universal primers UP1 and UP2 comprise different labels ( [symbol] or ♦).

In the embodiments shown in FIG. 2, allele-specific probe ASP1 comprises a pivotal nucleotide that Is complementary to the nucleotide at the SNP locus. This probe hybridizes to the SNP locus, and the second probe hybridizes immediately adjacent to it. Under ligation conditions, these probes-are ligated to each other. The ligation product is then amplified by the universal primers UP1 and UP3. In certain embodiments, the foregoing procedure may be adapted so that an oligonucleotide ligation reaction is performed on multiple SNP loci in the same reaction mixture. In certain such embodiments, the resulting ligation products are amplified by the universal primers UP1 and UP3 or by UP2 and UP3.

In the embodiments shown in FIG. 2, at least a portion of the STR amplification reaction mixture is combined with at least a portion of the oligonucleotide ligation and PCR reaction mixture. The combined reaction mixture is then subjected to CE in a single capillary channel, and the labels are detected. The rate at which the STR amplification products migrate through the channel is a function of their size. The size of the STR amplification products and the color of their labels identify the STR allele(s) at each STR locus. The rate at which the amplified ligation product migrates through the channel is also a function of its size. The color of the label attached to the amplified ligation product identifies the SNP allele present at the SNP locus. In the embodiments shown in FIG. 2, the size of the amplified ligation product does not overlap with the size of the STR amplification products STR1 and STR4, which comprise the same label as the amplified ligation product. Furthermore, the size of STR amplification products from different STR loci that comprise the same label (e.g., the amplification products of STR2 and STR6) do not overlap in size.

In certain embodiments, the labels that are detected are displayed in a single output. Thus, in certain embodiments, the STR alleles and SNP alleles may be identified by referring to a single output, even though the STR amplication reaction and the oligonucleotide ligation and PCR reaction took place in separate reaction mixtures.

In certain embodiments, a kit for analyzing a plurality of STR loci and at least one SNP locus in a sample, comprising nucleic acid is provided. In certain embodiments, a kit is provided comprising any of the components used in any of the methods described above for analyzing STR and SNP loci. In certain embodiments, the kit comprises a plurality of STR-specific primer sets and at least one primer that selectively hybridizes to a SNP locus. In certain embodiments, the kit further comprises at least one universal primer comprising a label. In certain embodiments, the at least one primer that selectively hybridizes to a SNP locus is an allele-specific primer. In certain embodiments, the plurality of STR-specific primer sets and the at least one primer that selectively hybridizes to a SNP locus are capable of generating detectable amplification products in a single reaction mixture. In certain such embodiments, a plurality of STR alleles and at least one SNP allele are identified using a single reaction mixture. In certain embodiments, the plurality of STR-specific primer sets and the at least one primer that selectively hybridizes to a SNP locus generate amplification products that are detectable in a single output, thus allowing identification of a plurality of STR alleles and at least one SNP allele by referring to a single output. In certain embodiments, amplification products from different loci do not overlap in size. In certain embodiments, amplification products from a given locus may overlap in size with amplification products from one or more different loci. In certain such embodiments, amplification products from different loci comprise different labels.

In certain embodiments, a kit comprises a plurality of STR-specific primer sets and at least one probe that selectively hybridizes to a SNP locus. In certain embodiments, the kit further comprises at least one universal primer comprising a label. In certain embodiments, the at least one probe that selectively hybridizes to a SNP locus is an allele-specific probe. In certain embodiments, the plurality of STR-specific primer sets and the at least one probe that selectively hybridizes to a SNP locus allow identification of a plurality of STR alleles and at least one SNP allele in a single output. In certain embodiments, the at least one probe that selectively hybridizes to a SNP locus comprises at least one allele-specific probe and a second probe suitable for use in an oligonucleotide ligation assay.

### C. EXAMPLE

In certain embodiments, a sample to be genotyped is combined with STR-specific primer sets that amplify the TH01, TPOX, CSF1PO, vWA, D3S1358, D7S820, D13S317, P16S539, D8S1179, D18S51, D21S11, D2S1338, and D19S433 loci. Such primer sets are available, e.g., in the AmpFLSTR Identifier® PCR amplification kit (Applied Biosystems, Foster City, Calif.). One primer from each of the primer sets that amplify D8S1179, D21S11, D7S820, and CSF1PO is labeled with the 6-FAM™ fluorescent label. One primer from each of the primer sets that amplify D3S1358, TH01, D13S317, D16S539, and D2S1338 is labeled with the VIC® fluorescent label. One primer from each of the primer sets that amplify D19S433, vWA, TPOX, and D18S51 is labeled with the NED™ fluorescent label.

In the same reaction mixture, the sample is combined with at least one first set of primers for identifying an allele at a biallelic SNP. The at least one first set of primers is selected from the following primer sets in Table 1, which are based on sequences of SNP loci at world wide website cstl.nist.gov/div831/strbase/SNP.htm:

TABLE 1

| Primer | SNP (by | | | |
|---|---|---|---|---|
| set # | TSC #) | Primers | Sequence (5′ to 3′) | SEQ ID NO: |
| 1 | 0252540 | ASP1-T | GCCTCGACCGCTCCTCCAGCGACGGGAAACTGCTGGGTCaGT | SEQ ID NO: 1 |
| | | ASP1-C | GCCTCGACCGCTCCTCCAGCGACTTTTTGGGAAACTGCTGGGTCcGC | SEQ ID NO: 2 |
| | | S1 | CTCCTCCGCCTGCCACCGTGCCAATGACCTGCCCCACAGGAG | SEQ ID NO: 3 |
| 2 | 1342445 | ASP2-A | GCCTCGACCGCTCCTCCAGCGACGGGAGACAGGCCCATaCA | SEQ ID NO: 4 |
| | | ASP2-T | GCCTCGACCGCTCCTCCAGCGACTTTTTGGGAGACAGGCCCATcCT | SEQ ID NO: 5 |
| | | S2 | CTCCTCCGCCTGCCACCGTGCCGCCATTCAGAACTAACTAGTCTGGGA | SEQ ID NO: 6 |
| 3 | 1156239 | ASP3-A | GCCTCGACCGCTCCTCCAGCGACCAGAAAAGGCAGGAACCTGGcCA | SEQ ID NO: 7 |
| | | ASP3-T | GCCTCGACCGCTCCTCCAGCGACTTTTTCAGAAAAGGCAGGAACCTGGtCT | SEQ ID NO: 8 |
| | | S3 | CTCCTCCGCCTGCCACCGTGCCCGACGGGGGTTGAGTGGTTCAG | SEQ ID NO: 9 |
| 4 | 0846740 | ASP4-G | GCCTCGACCGCTCCTCCAGCGACACCAACCCCACAAAGCcGG | SEQ ID NO: 10 |
| | | ASP4-C | GCCTCGACCGCTCCTCCAGCGACTTTTTACCAACCCCACAAAGCtGC | SEQ ID NO: 11 |
| | | S4 | CTCCTCCGCCTGCCACCGTGCCATTAGAGCAGCCAAGTCCTGACCA | SEQ ID NO: 12 |
| 5 | 0421788 | ASP5-G | GCCTCGACCGCTCCTCCAGCGACGATGCCTCTTGCATTGTGAcCG | SEQ ID NO: 13 |
| | | ASP5-C | GCCTCGACCGCTCCTCCAGCGACTTTTTGATGCCTCTTGCATTGTGAtCC | SEQ ID NO: 14 |
| | | S5 | CTCCTCCGCCTGCCACCGTGCCGCTCAACAGCACAACTCTGCTACAGC | SEQ ID NO: 15 |
| 6 | 1588825 | ASP6-A | GCCTCGACCGCTCCTCCAGCGACGAGCCAAGAATCGCAGGaAA | SEQ ID NO: 16 |
| | | ASP6-T | GCCTCGACCGCTCCTCCAGCGACTTTTTGAGCCAAGAATCGCAGGcAT | SEQ ID NO: 17 |
| | | S6 | CTCCTCCGCCTGCCACCGTGCCGCTAAAGCAGCTCTGAAACCCA | SEQ ID NO: 18 |

The amplification products of a given primer set differ in size from those of any other primer set.

The allele-specific primers in a given primer set are designated as “ASP” followed by the number of the primer set and the identity of the pivotal nucleotide. The second primer in a given primer set is designated as S followed by the number of the primer set. The first 23 nucleotides of all the allele-specific primers are identical in sequence. That sequence does not hybridize to any of the SNP loci. That sequence is the same as the sequence of a first universal primer (UP1). In certain embodiments, the allele-specific primers may lack those first 23 nucleotides. In certain such embodiments, the allele-specific primers are labeled with PET.

In ASP1-C, ASP2-T, ASP3-T, ASP4-C, ASP5-C, and ASP6-T, a string of five T's follows the first 23 nucleotides. Because of that string of five T's, the amplification product generated by ASP1-C, ASP2-T, ASP3-T, ASP4-C, ASP5-C, or ASP6-T is larger than the amplification product generated by the other allele-specific primer in the same primer set. Thus, the size of the amplification product indicates which SNP allele is present at a particular SNP locus.

The 3′ portions of the allele-specific primers hybridize to their respective SNP loci. The nucleotide at the 3′ end of the allele-specific primers is complementary to one of the two possible nucleotides at the polymorphic site. Additionally, the third nucleotide from the 3′ end of the allele-specific primers (lowercase) is a mismatch with respect to the corresponding nucleotide at the target SNP locus. That mismatch Is introduced to Improve the specificity of the amplification. See, e.g., Papp et al (2003) BioTechniques 34:1068-1072; and Okimoto et al. (1996) BioTechniques 21:20-26. In various embodiments, such a mismatch could be introduced at any position in the portion of an allele-specific primer that hybridizes to a target SNP locus.

The first 22 nucleotides of all of the second primers are identical in sequence. That sequence does not hybridize to any of the SNP loci. Instead, that sequence is identical to the second of two universal primers (UP2). The nucleotides that follow the first 22 nucleotides of the second primers hybridize to their respective SNP loci.

The sequences of the universal primers are given below. Each universal primer is labeled with the PET® fluorescent label:

| Primer | (Label)-Sequence (5′ to 3′) | SEQ ID NO: |
|---|---|---|
| UP1 | (PET)- | SEQ ID NO: 19 |
| | GCCTCGACCGCTCCTCCAGCGAC | |
| UP2 | (PET)- | SEQ ID NO: 20 |
| | CTCCTCCGCCTGCCACCGTGCC | |

The reaction mixture is then subjected to the polymerase chain reaction. Amplification products are generated from the STR loci and the at least one SNP locus, with the labeled primers becoming Incorporated into the amplification products. STR amplification products are labeled with either the 6-FAM™, VIC®, or NED™ fluorescent label. SNP amplification products are labeled with the PET® fluorescent label. All or a portion of the reaction mixture is subjected to CE in a single capillary channel. The labels are detected and displayed in a single output. The rate at which the STR amplification products migrate through the channel is a function of their size. The size of the STR amplification products and the color of their labels Identify the STR allele(s) at each STR locus. The rate at which the SNP amplification products migrate through the channel is also a function of their size, which identifies the SNP allele(s) present at the at least one SNP locus.

What is claimed is:

1. A method of genotyping a sample comprising nucleic acid, the method comprising analyzing a plurality of STR loci in the sample and analyzing at least one SNP locus in the sample, thereby genotyping the sample.

2. The method of claim 1, wherein the plurality of STR loci comprises one or more CODIS STR loci.

3. The method of claim 1, wherein the analyzing a plurality of STR loci comprises using PCR to generate a plurality of PCR products.

4. The method of claim 3, wherein the size of at least two of the plurality of PCR products indicates the identity of at least two STR alleles.

5. The method of claim 1, wherein the analyzing a plurality of STR loci comprises:
combining at least a portion of the sample with a plurality of STR-specific primer sets, wherein an STR-specific primer set comprises a first primer and a second primer for amplifying an STR locus; and
subjecting the sample to amplification.

6. The method of claim 5, wherein at least one of the primers in the plurality of STR-specific primer sets further comprises a label.

7. The method of claim 1, wherein the analyzing a plurality of STR loci and the analyzing at least one SNP locus comprise processes that occur in separate reaction mixtures.

8. The method of claim 7, wherein the analyzing a plurality of STR loci and the analyzing at least one SNP locus further comprise combining the separate reaction mixtures to form a combined reaction mixture.

9. The method of claim 8, wherein the analyzing a plurality of STR loci and the analyzing at least one SNP locus further comprise detecting in the combined reaction mixture one or more labels that identify a plurality of STR alleles and at least one SNP allele in a single output.

10. The method of claim 1, wherein the at least one SNP locus provides information on phenotype.

11. The method of claim 1, wherein the analyzing at least one SNP locus comprises combining at least a portion of the sample with at least one allele-specific primer and subjecting the at least a portion of the sample to an extension assay.

12. The method of claim 11, wherein the analyzing at least one SNP locus comprises using allele-specific PCR or an allele-specific primer extension assay.

13. The method of claim 11, wherein the analyzing at least one SNP locus comprises using an allele-specific nucleotide incorporation assay.

14. The method of claim 13, wherein the analyzing at least one SNP locus comprises using a single base extension assay.

15. The method of claim 1, wherein the analyzing at least one SNP locus comprises combining at least a portion of the sample with at least one allele-specific probe and detecting hybridization of the at least one allele-specific probe to the SNP locus.

16. The method of claim 15, wherein the analyzing at least one SNP locus comprises using a method selected from an allele-specific oligonucleotide hybridization assay, a 5′ nuclease assay, an assay employing molecular beacons, an assay employing flap endonuclease, and an oligonucleotide ligation assay.

17. The method of claim 1, wherein the analyzing a plurality of STR loci and the analyzing at least one SNP locus occur in the same reaction mixture.

18. The method of claim 17, wherein the analyzing a plurality of STR loci and the analyzing at least one SNP locus comprise using PCR.

19. The method of claim 18, wherein the analyzing at least one SNP locus comprises using allele-specific PCR.

20. A kit for analyzing a plurality of STR loci and at least one SNP locus in a sample comprising nucleic acid, wherein the kit comprises a plurality of STR-specific primer sets and at least one primer that selectively hybridizes to a SNP locus.

21. The kit of claim 20, further comprising at least one universal primer comprising a label.

22. The kit of claim 20, wherein the at least one primer that selectively hybridizes to a SNP locus is an allele-specific primer.

23. The kit of claim 20, wherein the plurality of STR-specific primer sets and the at least one primer that selectively hybridizes to a SNP locus are capable of generating detectable amplification products in a single reaction mixture, wherein the amplification products indicate the identity of a plurality of STR alleles and at least one SNP allele.

24. The kit of claim 20, wherein the plurality of STR-specific primer sets and the at least one primer that selectively hybridizes to a SNP locus generate amplification products that are detectable in a single output, wherein the amplification products indicate the identity of a plurality of STR alleles and at least one SNP allele.

25. The kit of claim 24, wherein amplification products from different loci do not overlap in size.

26. The kit of claim 24, wherein amplification products from different loci overlap in size.

27. The kit of claim 26, wherein amplification products that overlap in size further comprise different labels.

28. A kit for analyzing a plurality of STR loci and at least one SNP locus in a sample comprising nucleic acid, wherein the kit comprises a plurality of STR-specific primer sets and at least one probe that selectively hybridizes to a SNP locus.

29. The kit of claim 28, further comprising at least one universal primer comprising a label.

30. The kit of claim 28, wherein the at least one probe that selectively hybridizes to a SNP locus is an allele-specific probe.

31. The kit of claim 28, wherein the at least one probe that selectively hybridizes to a SNP locus comprises at least one allele-specific probe and a second probe suitable for use in an oligonucleotide ligation assay.

32. The kit of claim 28, wherein the plurality of STR-specific primer sets and the at least one probe that selectively hybridizes to a SNP locus allow identification of a plurality of STR alleles and at least one SNP allele in a single output.


SEQUENCE LISTING (20 sequences)

SEQ ID NO: 1 - DNA, 42 bp, Artificial Sequence
  Synthetic Primer
gcctcgaccgctcctccagcgacgggaaactgctgggtcagt

SEQ ID NO: 2 - DNA, 47 bp, Artificial Sequence
  Synthetic Primer
gcctcgaccgctcctccagcgactttttgggaaactgctgggtccgc

SEQ ID NO: 3 - DNA, 42 bp, Artificial Sequence
  Synthetic Primer
ctcctccgcctgccaccgtgccaatgacctgccccacaggag

SEQ ID NO: 4 - DNA, 41 bp, Artificial Sequence
  Synthetic Primer
gcctcgaccgctcctccagcgacgggagacaggcccataca

SEQ ID NO: 5 - DNA, 46 bp, Artificial Sequence
  Synthetic Primer
gcctcgaccgctcctccagcgactttttgggagacaggcccatcct

SEQ ID NO: 6 - DNA, 48 bp, Artificial Sequence
  Synthetic Primer
ctcctccgcctgccaccgtgccgccattcagaactaactagtctggga

SEQ ID NO: 7 - DNA, 46 bp, Artificial Sequence
  Synthetic Primer
gcctcgaccgctcctccagcgaccagaaaaggcaggaacctggcca

SEQ ID NO: 8 - DNA, 51 bp, Artificial Sequence
  Synthetic Primer
gcctcgaccgctcctccagcgactttttcagaaaaggcaggaacctggtct

SEQ ID NO: 9 - DNA, 44 bp, Artificial Sequence
  Synthetic Primer
ctcctccgcctgccaccgtgcccgacgggggttgagtggttcag

SEQ ID NO: 10 - DNA, 42 bp, Artificial Sequence
  Synthetic Primer
gcctcgaccgctcctccagcgacaccaaccccacaaagccgg

SEQ ID NO: 11 - DNA, 47 bp, Artificial Sequence
  Synthetic Primer
gcctcgaccgctcctccagcgactttttaccaaccccacaaagctgc

SEQ ID NO: 12 - DNA, 46 bp, Artificial Sequence
  Synthetic Primer
ctcctccgcctgccaccgtgccattagagcagccaagtcctgacca

SEQ ID NO: 13 - DNA, 45 bp, Artificial Sequence
  Synthetic Primer
gcctcgaccgctcctccagcgacgatgcctcttgcattgtgaccg

SEQ ID NO: 14 - DNA, 50 bp, Artificial Sequence
  Synthetic Primer
gcctcgaccgctcctccagcgactttttgatgcctcttgcattgtgatcc

SEQ ID NO: 15 - DNA, 48 bp, Artificial Sequence
  Synthetic Primer
ctcctccgcctgccaccgtgccgctcaacagcacaactctgctacagc

SEQ ID NO: 16 - DNA, 43 bp, Artificial Sequence
  Synthetic Primer
gcctcgaccgctcctccagcgacgagccaagaatcgcaggaaa

SEQ ID NO: 17 - DNA, 48 bp, Artificial Sequence
  Synthetic Primer
gcctcgaccgctcctccagcgactttttgagccaagaatcgcaggcat

SEQ ID NO: 18 - DNA, 44 bp, Artificial Sequence
  Synthetic Primer
ctcctccgcctgccaccgtgccgctaaagcagctctgaaaccca

SEQ ID NO: 19 - DNA, 23 bp, Artificial Sequence
  Synthetic Primer
gcctcgaccgctcctccagcgac

SEQ ID NO: 20 - DNA, 22 bp, Artificial Sequence
  Synthetic Primer
ctcctccgcctgccaccgtgcc